(19) United States
(12) Patent Application Publication
(10) Pub. No.: US 2023/0272328 A1
(43) Pub. Date: Aug. 31, 2023

(54) BIOREACTOR ASSEMBLIES, PERFUSION BIOREACTOR SYSTEMS, AND METHODS

(71) Applicant: Advanced Solutions Life Sciences, LLC, Lousiville, KY (US)

(72) Inventors: Justin Palmer, Louisville, KY (US); Scott Cambron, Louisville, KY (US); James B. hOYING, Manchester, NH (US); Michael Golway, Louisvile, KY (US)

(21) Appl. No.: 18/175,144

(22) Filed: Feb. 27, 2023

Related U.S. Application Data
(60) Provisional application No. 63/314,212, filed on Feb. 25, 2022.

(51) Int. Cl. C12M 1/00; C12M 1/36; C12M 1/12; C12M 1/34; C12M 3/00

(52) U.S. Cl. CPC C12M 29/10; C12M 41/48; C12M 25/00; C12M 41/36; C12M 41/44; C12M 21/08

(57) ABSTRACT

The present disclosure relates to a bioreactor assembly having a housing defining an interior chamber; a lid assembly removably coupled to the housing and enclosing the interior chamber; and a gimbal assembly disposable within the interior chamber. The gimbal assembly includes a cradle configured to hold an organ or organ scaffold and an arm assembly configured to move the cradle between a plurality of positions. The bioreactor assembly also includes an ultrasound imaging unit positioned to capture volumetric and/or spatial data of the organ or organ scaffold.

### CROSS REFERENCE TO RELATED APPLICATIONS

This application claims benefit of priority of U.S. Provisional Patent Application No. 63/314,212 filed on Feb. 25, 2022, entitled, “Organ Perfusion Bioreactor and Methods of Use in Regenerative Medicine,” the content of which is hereby incorporated by reference in its entirety.

### TECHNICAL FIELD

The present specification relates generally to bioreactor assemblies, perfusion bioreactor systems, and methods, and more particularly to bioreactor assemblies, perfusion bioreactor systems, and methods for production of recellularized organs for use in regenerative medicine.

### BACKGROUND

The field of whole-organ tissue engineering has been revolutionized since the 2008 publication of the first perfusion-decellularized whole heart and subsequent efforts to recellularize the scaffold to generate a transplantable organ. The manufacture of such organs currently involves manual approaches whereby a trained expert executes each fabrication and processing step.

Accordingly, a need exists for devices, systems, and methods to provide improved processing and fabrication.

### SUMMARY

Embodiments of the present disclosure are directed to improvement of processing structures, systems, and methods for tissue manufacture as will be described in greater detail herein.

In one embodiment, the present disclosure relates to a bioreactor assembly comprising: a housing defining an interior chamber; a lid assembly removably coupled to the housing and enclosing the interior chamber; a gimbal assembly disposable within the interior chamber, the gimbal assembly comprising: a cradle configured to hold an organ or organ scaffold; and an arm assembly configured to move the cradle between a plurality of positions; and an ultrasound imaging unit positioned to capture volumetric and/or spatial data of the organ or organ scaffold.

In another embodiment, the present disclosure relates to a perfusion bioreactor system comprising: a bioreactor comprising: a housing defining an interior chamber; a lid assembly removably coupled to the housing and enclosing the interior chamber and comprising one or more inlet ports and one or more outlet ports; a gimbal assembly disposable within the interior chamber, the gimbal assembly comprising: a cradle configured to hold an organ or organ scaffold; and an arm assembly configured to move the cradle between a plurality of positions; and an ultrasound imaging unit positioned to capture volumetric and/or spatial image data of the organ or organ scaffold; a fluid perfusion system, fluidically coupled to the bioreactor, comprising: a fluid reservoir configured to hold a perfusate; and a pump fluidically coupling the one or more inlet ports to the fluid reservoir; an injection assembly configured to dispense material to the organ or organ scaffold within the chamber; one or more processors communicatively coupled to the injection assembly, the ultrasound imaging unit, and the gimbal system, one or more memory modules communicatively coupled to the one or more processors, and machine-readable instructions stored on the one or more memory modules that, when executed by the one or more processors, cause the perfusion bioreactor system to: collect volumetric and/or spatial image data of the organ or organ scaffold using the ultrasound imaging unit; identify one or more initiation locations on the organ or organ scaffold within the volumetric and/or spatial image data; position the organ or organ scaffold using the gimbal assembly such that the one or more initiation locations are aligned with the injection assembly.

In yet another embodiment, the present disclosure relates to a method of recellularizing an organ scaffold, the method comprising placing the organ within a bioreactor assembly; collecting, with a processor, volumetric and/or spatial image data of the organ scaffold using an ultrasound imaging unit communicatively coupled to the processor; processing, with the processor, the volumetric and/or spatial image data to identify one or more initiation locations; controllably adjusting the bioreactor assembly communicatively coupled to the processor with the processor to position the organ scaffold within the to provide access to the one or more initiation locations; and dispensing or injecting a cellular material onto or into the organ scaffold.

The and additional features provided by the embodiments described herein will be more fully understood in view of the following detailed description in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A depicts an illustrative bioreactor assembly according to one or more embodiments shown and described herein;

FIG. 1B depicts a cross-sectional view of the bioreactor assembly of FIG. 1A according to one or more embodiments shown and described herein;

FIG. 1C depicts a different cross-sectional view of the bioreactor assembly of FIG. 1A according to one or more embodiments shown and described herein;

FIG. 2A depicts a top perspective view of an illustrative lid assembly for use with a bioreactor assembly according to one or more embodiments shown and described herein;

FIG. 2B depicts a bottom perspective view of an illustrative lid assembly for use with a bioreactor assembly according to one or more embodiments shown and described herein;

FIG. 3A depicts an illustrative gimbal assembly for disposing within a bioreactor assembly according to one or more embodiments shown and described herein;

FIG. 3B depicts the gimbal assembly of FIG. 3A indicating respective axes of rotation, according to one or more embodiments shown and described herein;

FIG. 4A depicts an illustrative drive system of the gimbal assembly of FIG. 3A, according to one or more embodiments shown and described herein;

FIG. 4B depicts the drive system of FIG. 4A coupled to one or more joint assemblies, according to one or more embodiments shown and described herein;

FIG. 4C depicts another view of the drive system of FIG. 4B coupled to the one or more joint assemblies, according to one or more embodiments shown and described herein;

FIG. 4D depicts another view of the drive system of FIG. 4C, with a joint assembly transparently depicted, according to one or more embodiments shown and described herein;

FIG. 5 depicts an illustrative perfusion bioreactor system, according to one or more embodiments shown and described herein;

FIG. 6 depicts a perfusion bioreactor system housed within a three dimensional bioprinter apparatus, according to one or more embodiments shown and described herein;

FIG. 7A depicts a schematic flow path of a perfusion system for use with a perfusion bioreactor system, according to one or more embodiments shown and described herein;

FIG. 7B depicts an organ cannulated and coupled to the perfusion bioreactor system, according to one or more embodiments shown and described herein;

FIG. 8 depicts a schematic diagram of an illustrative perfusion bioreactor system, according to one or more embodiments shown and described herein;

FIG. 9 depicts a flowchart for a method of using a perfusion bioreactor system, according to one or more embodiments shown and described herein;

FIG. 10 depicts a flowchart summarizing the stages of generating a bioengineered organ for transplantation, according to one or more embodiments shown and described herein; and

FIG. 11 depicts an illustrative method for transporting a bioreactor assembly through various stages of bioengineering, according to one or more embodiments shown and described herein.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

Embodiments described herein are generally directed to bioreactor assemblies and perfusion bioreactor systems for use in bioengineering organs for regenerative medicine, and methods of using the same. The bioreactor assemblies as described herein generally include a gimbal assembly configured for 3-dimensional movement and an ultrasound imaging unit. In operation, bioreactor assemblies according to the present disclosure are capable of supporting an organ through various stages of bioengineering, such as, but not limited to decellularization, recellularization, maturation, and/or functional testing. To assist in the various stages of bioengineering, embodiments of the present disclosure provide improved procedures and process control. For example, bioreactor assemblies according to the present disclosure may support and position an organ or organ scaffold for material injection, testing, imaging, etc., while maintaining a sterile environment. Accordingly, in embodiments, various processing steps may be automated using the bioreactor assembly and/or the perfusion bioreactor system leading to improved organ generation and consistent results, for example. These and other features and embodiments of the bioreactors are disclosed in greater detail herein with reference to the appended figures.

As used herein, the term “subject” and “recipient” are interchangeable and refer to any animal or human subject for which an organ transplant may be desirable. Illustrative, non-limiting examples of organs for use in embodiments of the present disclosure include, but are not limited to, heart, kidney, liver, lung, pancreas, and intestines, or portions thereof (e.g., heart valves, lung lobes, etc.).

In some embodiments, an organ, organ scaffold, or portion thereof may be allogenic, autologous, or xenogeneic to the subject. In some embodiments, the organ or organ scaffold may comprise synthetic components. In some embodiments, the organ or organ scaffold may be obtained from non-human mammals. Illustrative, non-limiting examples include, non-human primates, such as baboons or chimpanzees, rodents, pigs, rabbits, cattle, sheep, dogs, cats, and cows. In some embodiments, the organ or organ scaffold may be obtained from a human source, such as a cadaver or living-donor.

In operation, bioreactor assemblies and perfusion bioreactor systems according to the present disclosure may be capable of supporting an organ through various stages of bioengineering, including decellularization, recellularization, maturation, and/or functional testing. It is contemplated, that bioreactor assemblies and perfusion bioreactor systems may support an organ through various stages of bioengineering including, but not limited to, decellularization, recellularization, maturation, and functional testing. It is also contemplated that bioreactor assemblies and perfusion bioreactor systems according to the present disclosure can be used to automate any combination of the stages of bioengineering, such as those disclosed in U.S. Pat. No. 11,414,644, filed Jan. 29, 2019, and entitled “Methods of Recellularizing a Tissue or Organ for Improved Transplantability,” U.S. Pat. No. 10,233,420, filed Sep. 1, 2022, and entitled “Methods of Recellularizing a Tissue or Organ for Improved Transplantability,” U.S. Pat. No. 10,441,609, filed Dec. 21, 2012, and entitled “Decellularization and Recellularization of Solid Organs,” U.S. Pat. No. 10,220,056, filed Jun. 10, 2013, and entitled “Decellularization and Recellularization of Solid Organs,” U.S. Patent Publication No. 2022/0062349, filed Aug. 4, 2021, and entitled “Decellularization and Recellularization of Organs and Tissues,” and U.S. Patent Publication No. 2023/0002723, filed Jul. 6, 2022, and entitled “Methods of Recellularizing a Tissue or Organ for Improved Transplantability,” the contents of which are incorporated herein by reference in their entirety. Additional information regarding the various stages can be found, for example, in Ott H C, Matthiesen T S, Goh S K, Black L D, Kren S M, Netoff T I, Taylor D A. Perfusion-decellularized matrix: using nature's platform to engineer a bioartificial heart. Nat Med. 2008 February; 14(2):213-21 and Tang-Quan K R, Mehta N A, Sampaio L C, Taylor D A. Whole Cardiac Tissue Bioscaffolds. Adv Exp Med Biol. 2018; 1098:85-114, the contents of which are hereby incorporated by reference in their entirety.

The term “decellularized” or “decellularization” as used herein may refer to an organ, or a portion thereof, from which the cellular and tissue content has been substantially removed, leaving behind an intact or substantially-intact, acellular scaffold. In embodiments, the removal of antigenic epitopes associated with cellular components minimizes or eliminates adverse immune responses (i.e., rejection) in a transplant recipient. In embodiments, this scaffold is comprised of a complex three-dimensional network of extracellular matrix (ECM). In embodiments, the scaffold may be rigid, semi-rigid, or flexible.

In embodiments, the three-dimensional network of ECM is composed primarily of collagen. However, other components may be present, including, but not limited to cytokines, proteoglycans, polysaccharides, elastin, fibronectin, integrin, laminin, fibrillin, endosomes, and/or extracellular bound vesicles. In embodiments, a decellularized organ provides a biocompatible scaffold onto which one or more cell types may be infused or injected.

As used herein, the term “recellularize” or “recellularization” as used herein refers to an engraftment or distribution of a plurality of cells, described in further detail below onto a decellularized organ scaffold. A recellularized organ, interchangeably used with “bioengineered organ” herein, may comprise morphology and/or activity of a native organ. Recellularization can comprise seeding, engraftment, and reendothelialization.

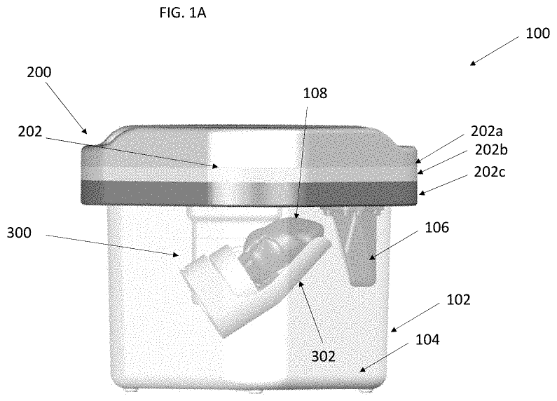

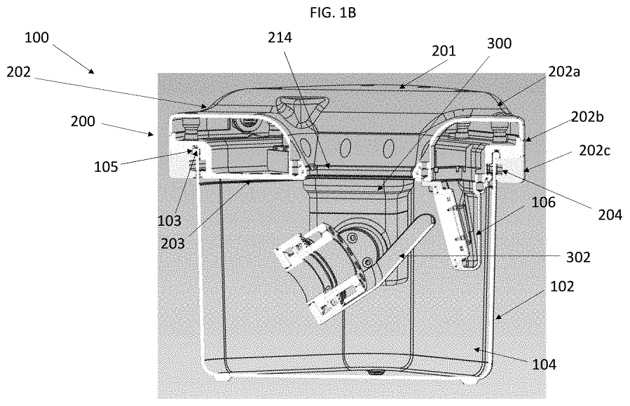

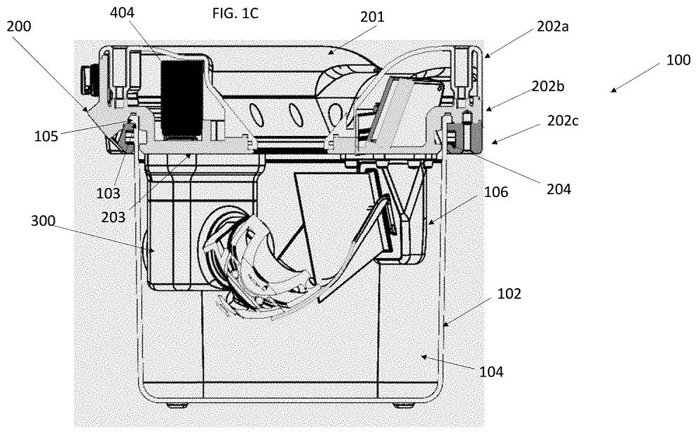

Referring now to FIG. 1A, an illustrative bioreactor assembly 100 is schematically depicted. In embodiments, the bioreactor assembly 100 includes a housing 102, a lid assembly 200, a gimbal assembly 300, and an ultrasound imaging unit 106. The bioreactor assembly 100 may include a greater or fewer number of components without departing from the scope of the present disclosure.

The housing 102 defines an interior chamber 104. For example, the interior chamber 104 is configured, e.g., sized and shaped, to hold an organ or organ scaffold 108 therein. In embodiments, the housing 102 may be rigid or semi-rigid and made of materials such as one or more of metals, plastics, foams, elastomers, ceramics, composites, combinations thereof, or the like. In embodiments, the housing 102 may be fabricated from an acoustically-transparent material. Suitable acoustically transparent materials include, but are not limited to polymethylpentene (PMP), polyimide, combinations thereof, and the like. In embodiments, the interior chamber 104 and any components disposed therein may be sterile or sterilizable via any conventional sterilization process.

Sterility of the bioreactor assembly 100 may be maintained using any suitable means, including, but not limited to, controlling and filtering airflow within and around an environment of the bioreactor assembly, filtering perfusate prior to delivery into the bioreactor assembly, and/or adding antibiotics, anti-fungals, and/or anti-microbials to the perfusate, discussed in greater detail herein.

Referring to FIGS. 1B-1C, cross-sections of the bioreactor assembly 100 are schematically depicted. The cross-section in FIG. 1B is taken along an imaging plane of the ultrasound imaging unit 106. In the illustrated embodiment, the lid assembly 200, generally includes a lid body 202 which may be formed from one or more layers coupled to one another. For example, the lid body 202 may include a first layer 202a, a second layer 202b, and a third layer 202c, though a greater or fewer number of layers are contemplated and possible. In embodiments, the first layer 202a provides a top surface 201 of the lid assembly 200, wherein the top surface 201 is exteriorly exposed. In embodiments, the second layer 202b provides a bottom surface 203 of the lid assembly 200, wherein the bottom surface 203 is exposed to the interior chamber 104 when assembled to the housing 102. The third layer 202c may be mounted to a periphery of the second layer 202b, such as surrounding the bottom surface of the lid second layer 202b. As will be described in greater detail herein, the lid body 202 may house various components of the bioreactor assembly 100 and/or define various fluid flow paths into and/or out of the bioreactor assembly 100. In embodiments, the lid assembly 200 may include one or more insulating layers to aide in thermal regulation of the chamber. In embodiments, the various layers of the lid assembly 200 may be coupled to one another via any conventional means, e.g., fasteners, clips, interlocks, press fittings, adhesives, O-rings, gaskets, seals, or the like. As will be described in greater detail below, various components may be housed within an interior volume of the lid assembly or otherwise coupled to the lid body 202.

Accordingly, and as should be understood, the bioreactor assembly 100 is configured such that the interior chamber 104 is accessible. For example, the housing 102 may have an open end to which the lid assembly 200 is removably coupled. For example, the lid assembly 200 may seal about a lip 103 of the housing 102. Referring collectively to FIGS. 1A and 1B, in embodiments, the lid assembly 200 includes a sealing mechanism 204 that allows the formation of an airtight and/or watertight seal between the lid assembly 200 and the housing 102, which may assist in maintaining suitable sterile conditions. Suitable sealing mechanisms include one or more connectors, latches, fasteners, O-rings, gaskets, seals, or the like to resealably close the lid assembly 200 to the housing 102. For example, as noted above, the third layer 202c may be mounted so as to be positioned around an outer periphery of the bottom surface. For example, the second layer 202b may include a recessed region into which the third layer 202c may be positioned such that a base surface of the third layer 202c, and the bottom surface of the second layer 202b are co-planar with one another. It is contemplated however, that the bottom surface of the second layer 202b and the base surface of the third layer 202c are not co-planar but are vertically offset from one another while still leaving the bottom surface of the second layer 202b unobstructed by the third layer 202c. In embodiments, between the third layer 202c and the periphery of the bottom surface 203 of the second layer 202b may be a gap sized to receive the lip 103 of the housing 102 therein. A side wall of the third layer 202c and a side wall of the second layer 202b may define lateral constraints of the gap, which may be sized to securely receive the lip. In some embodiments, the lip 103 may have a flange 105 that sits above the third layer 202c as depicted in FIG. 1B for further security. In embodiments, the third layer 202c and/or the second layer 202b, may include one or more O-ring or seal recesses for receiving an O-ring or other rubber seals therein to seal the lid assembly 200 to the housing 102. Accordingly, in embodiments, the lid assembly 200 hermetically seals to the housing 102, thereby maintaining the sterility of the interior chamber 104.

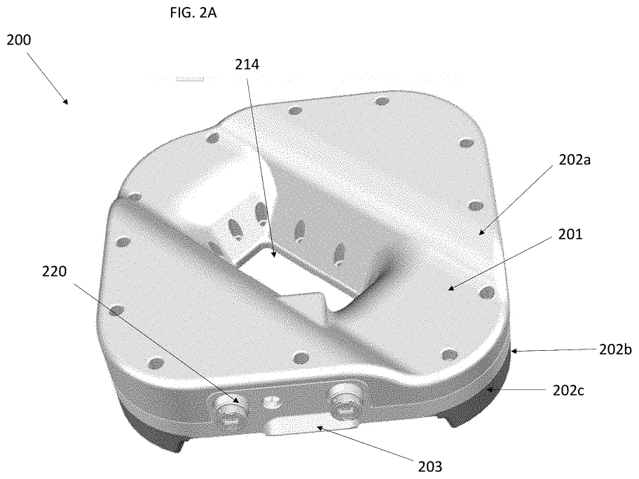

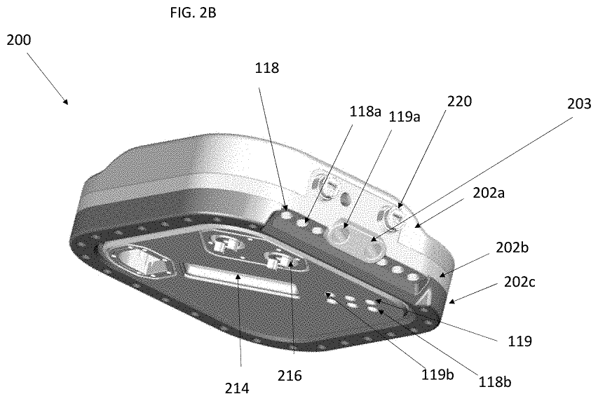

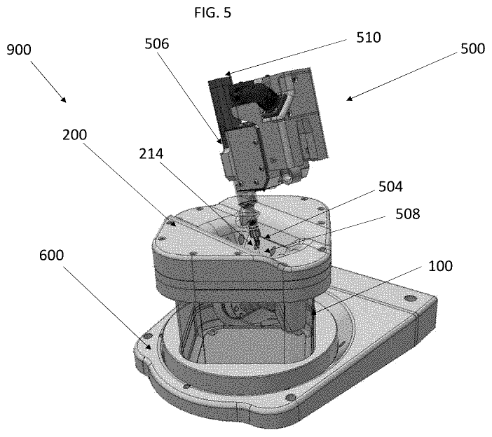

Referring now to FIGS. 1B and 2A, in embodiments, the lid assembly 200 defines an injection tool access port 214 for receiving an injection tool 504 (illustrated in FIG. 5) therethrough. For example, the lid body 202 may be recessed about the injection tool access port 214 such that the lid body 202 slopes toward the injection tool access port 214, which may be accessible via the top surface 201 of the first layer 202a of the lid body 202. In embodiments, the injection tool access port 214 extends throughout the one or more layers (i.e., 202a, 202b, and/or 202c) to provide access to the interior chamber 104. By providing an injection tool access port 214, the lid assembly 200 may remain in place throughout processes being performed within the interior chamber 104. In embodiments, the injection tool access port 214 is configured to open to allow access to the interior chamber 104. In embodiments, the injection tool access port 214 has a closed configuration preventing access into the interior chamber 104. The closed configuration may be achieved by any suitable means that renders the closed configuration watertight. In embodiments, the lid assembly 200 includes an automated door (e.g., hinged, sliding, or the like) which switches the injection tool access port 214 between the open configuration and the closed configuration. In embodiments, the automated door can be controllably opened and/or closed. In embodiments, the injection tool access port 214, while in a closed configuration may be covered by a pierceable membrane, such as a pierceable septum membrane, a self-healing membrane, or the like. In embodiments, the injection tool access port 214 can have a removable lid. In embodiments, including a removable lid, during automated processes the lid may be manipulated and/or removed as needed via a robotic arm (e.g., an appropriate end effector tool).

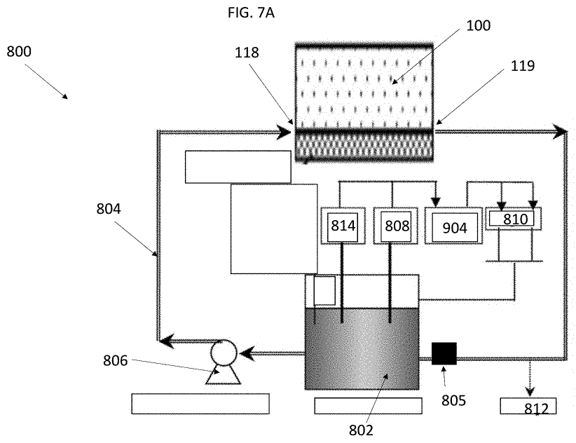

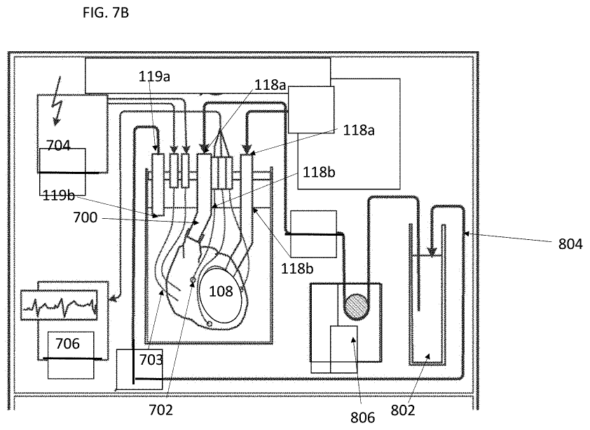

Referring now to FIGS. 2A, 2B, and 7B the bioreactor assembly 100 may include one or more inlet ports 118, such as exterior facing inlet ports 118a and interior chamber facing inlet ports 118b for introduction of fluid, such as perfusate, to the interior chamber 104. In embodiments, the exterior facing inlet ports 118a and interior chamber facing inlet ports 118b allow for the introduction of stimulating mechanisms, (e.g., electrodes 703, depicted in FIG. 7B) into the interior chamber 104. For example, and as shown the one or more exterior facing inlet ports 118a may be formed within the lid assembly 200, such as through a sidewall 203 (e.g., formed within the second layer 202b) of the lid body 202. In embodiments, the one or more inlet ports 118 may be used to position one or more sensors near the organ or organ scaffold 108, configured to measure specific physiologic parameters of the organ or organ scaffold 108 as the organ is undergoing recellularization processes or during maturation processes. For example, the bioreactor assembly 100 may comprise one or more sensors to measure biological activity of the organ or organ scaffold 108. Biological activity may include, but is not limited to, electrical activity, mechanical forces, contractions, ventricular wall motion, flow rate, pressure and stress of the organ or organ scaffold 108 or conditions of the bioreactor assembly 100. In embodiments, the biological activity of the cells on a recellularized organ or organ scaffold 108 may be monitored. In embodiments, pressure/flows within the organ/organ scaffold and/or outside of the organ/organ scaffold (e.g., internal to the bioreactor assembly) may also be monitored. Suitable measurements and monitoring can be found, for example, in Laboratory Textbook of Anatomy and Physiology (2001, Wood, Prentice Hall) and Current Protocols in Cell Biology (2001, Bonifacino et al., Eds, John Wiley & Sons), the contents of which are incorporated herein by reference.

In some embodiments, the one or more exterior facing inlet ports 118a may be angled and/or recessed with respect to the sidewall 203 of the lid body 202. Angled and/or recessed ports may improve ease of connecting fluid connection lines to the one or more exterior facing inlet ports 118a. As used herein, the terms “inlet” and “outlet” refer to the direction of perfusate flow relative to entering and exiting the interior chamber 104 of the bioreactor assembly 100. In embodiments, the lid assembly 200 defines one or more fluidic pathways therethrough from the exterior facing inlet port 118a into the interior chamber 104 of the housing 102, such as via one or more openings within the second layer 202b of the lid assembly 200. In other embodiments, it is contemplated that one or more inlet ports 118 may instead or in addition to being formed within the lid assembly 200, may be formed within the housing 102. In embodiments, the one or more fluid pathways, may be provided via tubing routed through the lid assembly 200.

In embodiments, the bioreactor assembly 100 may include one or more outlet ports 119, such as exterior facing outlet ports 119a and interior chamber facing outlet ports 119b for removal of fluid from the bioreactor. In embodiments, the one or more outlet ports 119 can allow for communication wires and/or recording mechanisms to transmit information from the organ or organ scaffold, such as EKG wires 702, shown in FIG. 7B. In embodiments, the lid assembly 200 includes an exterior facing outlet port 119a formed on an exterior facing surface of the lid assembly 200. For example, and as shown, the one or more exterior facing outlet ports 119a may be formed within the lid assembly, such as through a sidewall 203 of the lid body 202. In some embodiments, the one or more exterior facing outlet ports 119a may be angled and/or recessed with respect to the sidewall 203 of the lid body 202.

In embodiments, a fluidic pathway extends from an exterior facing outlet port 119a to an interior chamber facing outlet port 119b (e.g., which may be provided via tubing). In embodiments, the lid assembly 200 defines one or more fluidic pathways therethrough from the exterior facing outlet port 119a into the interior chamber 104 of the housing 102, such as via one or more openings within the second layer 202b of the lid assembly 200. In other embodiments, it is contemplated that one or more outlet ports 119 may instead or in addition to being formed within the lid assembly 200, be formed within the housing 102.

Each inlet port 118 and/or outlet port 119 of the bioreactor assembly 100 may include or be fluidically coupled to any suitable mechanism for initiating and conducting fluid flow through the port including, but not limited to, one or more peristaltic pumps, one or more pressurization mechanisms, and the like, which may be positioned within the lid assembly 200 or external to the lid assembly 200. In embodiments, the fluidic pathways formed within the lid assembly 200 couple the bioreactor assembly 100 to a perfusion system 800, discussed in greater detail below.

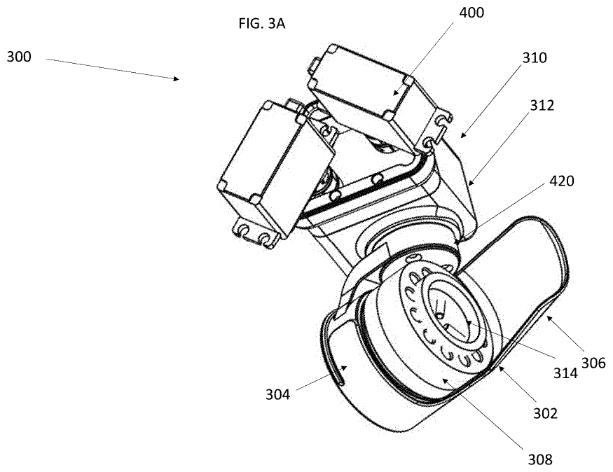

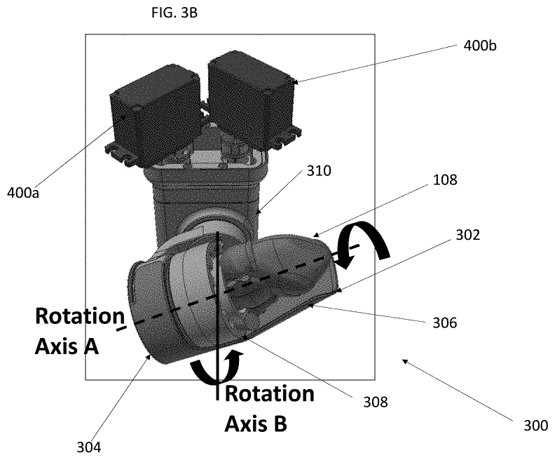

Referring jointly now to FIGS. 1A-1C and 3A-3B and as noted above, the bioreactor assembly 100 may also include a gimbal assembly 300. In embodiments, the gimbal assembly 300 is disposable within the interior chamber 104. In embodiments, the gimbal assembly 300 is coupled and sealed to the lid assembly 200. In embodiments, and as shown in FIG. 1B, the gimbal assembly 300 may be coupled to the lid assembly 200 such as to a bottom surface of the second layer 202b of the lid assembly 200, so as to be positioned within the interior chamber 104 when the lid assembly 200 is assembled to the housing 102. As shown in FIGS. 3A-3B, the gimbal assembly 300 generally includes a cradle 302 configured to hold an organ or organ scaffold 108 and an arm assembly 310 configured to move the cradle 302 between a plurality of positions, though a greater or fewer number of components are contemplated and possible.

In embodiments, the gimbal assembly 300 is configured to accommodate movement of an organ or organ scaffold 108 throughout one or more phases of bioengineering being carried out within the bioreactor assembly 100. For example, in embodiments, the gimbal assembly 300 is configured to rotate the organ or organ scaffold 108 between a plurality of positions to, for example, align one or more portion of the organ or organ scaffold 108 with the injection assembly access port 214 and/or the ultrasound imaging unit 106. Movements may also be related to specific positions for decellularization, recellularization, and/or maturation orientations. In embodiments, the gimbal assembly 300 may be configured to position the organ or organ scaffold 108 such that the transition between phases of bioengineering do not negatively impact the outcome of the bioengineered organ. For example, after decellularization, the gimbal assembly 300 may be configured to position the organ or organ scaffold 108 such that residual decellularization fluids are removed from the remaining organ scaffold 108, for example gravitationally, or by rinsing with perfusate. During recellularization procedures, the gimbal assembly 300 may be configured to rotate and position the organ scaffold 108 to aid in more robust and successful recellularization procedures, discussed in further detail below.

In embodiments, the gimbal assembly 300 may be controlled, such as via a processor executing non-transitory machine-readable instructions, described in further detail below, to mimic the natural anatomical positioning and support of the organ or organ scaffold 108. For example, during decellularization, the gimbal assembly 300 may position the organ or organ scaffold 108 in a substantially vertical position. In embodiments, the gimbal assembly 300 may be controlled, such as via a processor executing machine-readable instructions to change positions to prevent injury to the organ scaffold 108 and/or transition to a different phase of the bioengineering process. For example, during recellularization, the gimbal assembly 300 may position the organ or organ scaffold 108 in a horizontal position, such that it is supported through one or more injections, discussed in greater detail herein.

As shown in FIG. 3A the cradle 302 is coupled to the arm assembly 310 and generally includes a connector end 304 and a tissue support surface 306. The tissue support surface 306 may be configured to support the organ or organ scaffold 108 thereon in various positions. The connector end 304 is configured to couple the cradle 302 to the arm assembly 310. For example, the connector end 304 may couple the cradle 302 to one of the joint assemblies 420, described in further detail below. Any suitable means of coupling the connector end 304 to the arm assembly 310 is contemplated and possible, including adhesives, welding, fasteners, clips, magnets, etc. In embodiments, the cradle 302 is formed from any suitable material(s) to couple the cradle 322 to the arm assembly 310 and/or support the organ or organ scaffold 108. In embodiments, the suitable materials are biocompatible and/or autoclave compatible. Illustrative, non-limiting examples of suitable materials include glass, polymers, such as nylon, polyethylene (PE), polytetrafluoroethylene (PTFE), polypropylene, polyether ether ketone, PMP, silicone, metals, metal alloys, such as stainless steel, combinations thereof, and the like. In embodiments where the organ or organ scaffold 108 is more delicate, the cradle may be made from screens, nets or mesh structures, semi-solid gels, sponges, and the like.

In embodiments, the tissue support surface 306 extends from the connector end 304 and is positioned to support the organ or organ scaffold 108 thereon. In embodiments, the tissue support surface 306 may be generally curved to support the organ or organ scaffold 108 as it is moved using the arm assembly 310, discussed in further detail below. In embodiments, the tissue support surface 306 and the connector end 304 may be made from the same material. In embodiments, the tissue support surface 306 and the connector end 304 may be fabricated from different materials, depending on the needs of the specific organ or organ scaffold 108 and/or the stage of bioengineering.

In embodiments, the gimbal assembly 300 includes a tissue mount 308, configured to receive and support at least a portion of the organ or organ scaffold 108. In embodiments, the tissue mount 308 provides a transition between the arm assembly 310 and the cradle 302. In embodiments, the tissue mount 308 defines a channel 314 extending therethrough configured to receive tubing from the perfusion system 800 (discussed in greater detail below) to the tissue or organ scaffold 108. In embodiments, the tissue mount 308 is coupled to the cradle 302.

Referring again to FIG. 3A, the arm assembly 310 may include a mechanical housing 312, one or more drive systems 400, and one or more joint assemblies 420, though a greater or fewer number of components are contemplated and possible.

In embodiments, the arm assembly 310 includes a plurality of drive systems 400 which provide a multi-stage drive system. Although FIGS. 3A-3B depict a gimbal assembly 300 having two drive systems 400 in the arm assembly 310, any other number of drive systems 400 are contemplated and possible. Each drive system may interact, through any number of armatures, gears, etc., to rotate the cradle about a respective rotation axis, as shown in FIG. 3B.

As shown in FIG. 3B, the arm assembly includes a first drive system 400a configured to provide rotation about a first rotational axis (Rotation Axis A), and a second drive system 400b configured to provide rotation about a second rotational axis (Rotation Axis B). In embodiments, the first rotational axis (Rotation Axis A) and the second rotational axis (Rotation Axis B) can be positioned at any non-zero angle, relative to each other. In embodiments, the first rotational axis (Rotation Axis A) and/or the second rotational axis (Rotation Axis B) may move relative to one another. In embodiments, the first rotational axis (Rotation Axis A) and/or the second rotational axis (Rotation Axis B) may be stationary relative to one another.

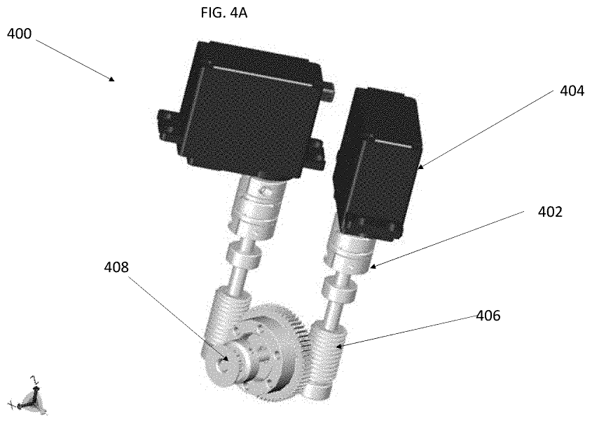

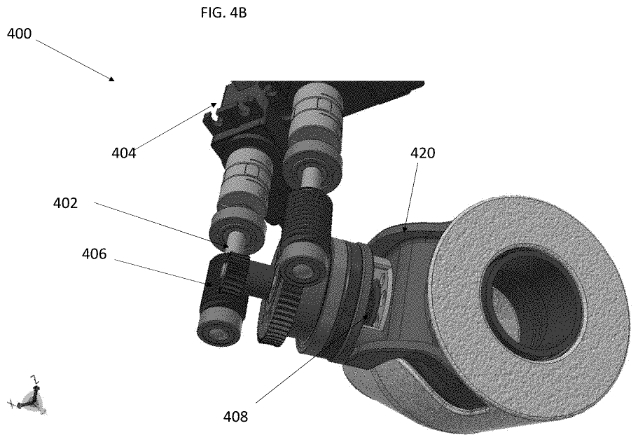

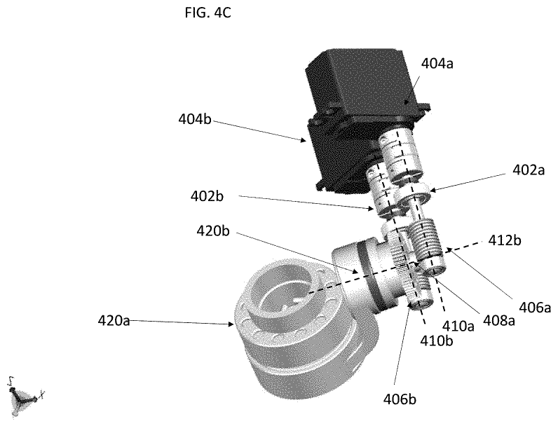

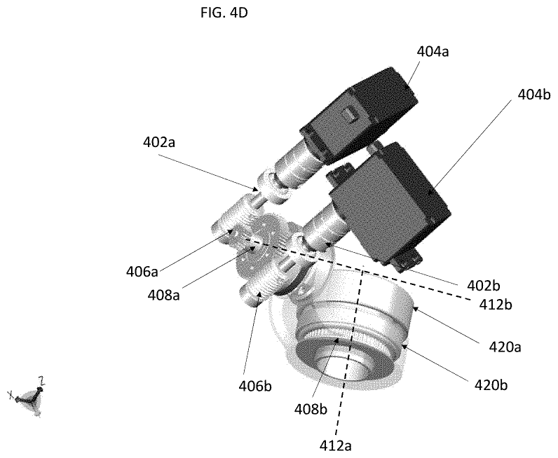

Referring jointly now to FIGS. 3A-3B and FIGS. 4A-4B, each drive system 400 generally includes a drive shaft 402 operably coupled to a motor 404 and a gear assembly 406, having one or more gears, mounted to the drive shaft 402. It is noted that a separate motor 404 is shown for each drive system, a single motor may be used which may be coupled to each drive shaft via any number of gears, pulleys, etc. In embodiments, such as shown in FIG. 4A, the gear assembly 406 includes worm gears, though other type gears are contemplated and possible. In embodiments, the gear assembly 406 is operatively coupled to a rotary assembly 408, such as a pulley, sprocket or additional gear assembly. The rotary assembly may be operatively coupled to a joint assembly 420 configured to provide movement of the organ or organ scaffold 108 around a rotational axis, as shown in FIG. 3B.

For example, and referring now to FIG. 4C, a first motor 404a is operably coupled to a first drive shaft 402a. A first gear assembly 406a is mounted to the first drive shaft 402a. The first gear assembly 406a rotates with the first drive shaft 402a around a rotational axis 410a. The first gear assembly 406a is operably coupled to a first rotary assembly 408a. In embodiments, such as illustrated in FIG. 4C, the first rotary assembly 408a includes a coupler, such as a dowel rod, which extends through a lumen of a second gear assembly 406b. The first rotary assembly 408a, for example, may include one or more timing belt pulleys coupled to one another and offset such that each rotate around a different rotational axis (e.g., 412a and 412b), different from the rotational axis 410a of the first drive shaft 402a. The first rotary assembly 408a is coupled to a first joint assembly 420a. The first joint assembly 420a is configured to allow rotation around the first rotational axis (Rotation Axis A, indicated in FIG. 3B), corresponding with rotational axis 412a.

Still referring to FIG. 4C, a second motor 404b is operably coupled to a second drive shaft 402b. A second gear assembly 406b is mounted to the second drive shaft 402b. The second gear assembly 406b rotates with the second drive 402b shaft around a rotational axis 410b. This rotational axis 410b can be parallel to rotational axis 410a, orthogonal to rotational axis 410a, or any angle with respect to rotational axis 410a. The second gear assembly 406a is coupled to a second joint assembly 420b, shown in greater detail in FIG. 4D. The second joint assembly 420b is configured to allow rotation around the second rotational axis (Rotation Axis B, indicated in FIG. 3B), corresponding with rotational axis 412b.

Referring back to FIGS. 3A-3B, the one or more drive systems 400 may be disposed, at least partially, within the mechanical housing 312. The mechanical housing 312 may provide a waterproof environment in which the drive systems 400 may operate. The mechanical housing 312 may be coupled to the lid assembly 200. As shown in FIG. 2, the lid assembly 200 may include one or more electrical connections 220 formed to interface with and electrically and communicatively couple the drive system 400 with the one or more processors 904, described in greater detail below.

In embodiments such as shown in FIG. 1C, the one or more motors 404 are disposed within the lid assembly 200 while the one or more drive shafts 402 are disposed within the mechanical housing 312. In such embodiments, the one or more motors 404 are operably coupled to a respective drive shaft 402 through an aperture 216 in a bottom surface of the lid assembly 200 and/or the mechanical housing 312.

Referring back to FIGS. 1A-1C, and as noted above, the bioreactor assembly 100 generally includes an ultrasound imaging unit 106. In embodiments, the ultrasound imaging unit 106 is disposed within the interior chamber 104 of the bioreactor assembly 100 and captures real- or near real-time ultrasound image data within the interior chamber 104. In embodiments, the ultrasound imaging unit 106 is coupled and sealed to the lid assembly 200, such as at the bottom surface 203 of the lid assembly 200 via any conventional techniques including use of adhesives, fasteners, seals, gaskets, O-rings, and the like. In embodiments, the ultrasound imaging unit 106 is capable of programmatically changing the amplitude, frequency, and duration of the ultrasound pulses, such as via control by a processor executing non-transitory computer readable instructions. In embodiments, some instrumentation and/or hardware (e.g., wiring, power sources, etc.) of the ultrasound imaging unit 106 may be housed within the lid assembly 200, as shown in FIG. 2B.

The ultrasound imaging unit 106 is configured to collect volumetric and/or spatial image data of the organ or organ scaffold 108 that is used to identify characteristics thereof. These characteristics will be used in the various stages of bioengineering, described in further detail herein. In embodiments, the ultrasound imaging plane is angled with respect to a horizontal axis, such as 10 degrees below the horizontal axis, 15 degrees below the horizontal axis, or the like to better capture ultrasound image data of an organ positioned within the cradle 302.

In embodiments, the bioreactor assembly 100 further includes one or more stimulating mechanisms to aid in maturation of the organ. Examples include electrodes 703 (depicted in FIG. 7B) to provide electrical stimuli for the organ. For example, electrodes 703 may be placed on an external surface of the organ or organ scaffold 108, such as at or near sinus nodes in the right atrium and/or ventricle of a heart. In embodiments, the stimulating mechanisms may be placed closer to a desired injection sited for improved electrical conductivity thru the organ or organ scaffold 108. As discussed above, the one or more inlet ports 118 may allow for the introduction of stimulating mechanisms, such as one or more electrodes 703. In embodiments, the electrodes 703 are coupled to an external pacemaker 704. The external pacemaker 704 may be configured to deliver electrical stimulation to the organ or organ scaffold 108 through the electrodes 703. In embodiment, the external pacemaker 704, such as via a control by a processor, may adjust the amplitude, voltage, and frequency of the electrical stimulation delivered to the organ or organ scaffold 108. In embodiments, the bioreactor assembly 100 may include one or more EKG wires 702 to transmit data related to the electrical activities of the heart to an EKG recorder 706.

Referring jointly now to FIGS. 5-8, a perfusion bioreactor system 900 is depicted. Perfusion bioreactors may generally include a bioreactor assembly 100 (such as described in detail above), an injection assembly 500, one or more bases 600, and a perfusion system 800 (shown in FIG. 7A), though a greater or fewer number of components are contemplated and possible. In embodiments, a bioreactor assembly 100 as previously described may be included as part of the perfusion bioreactor system 900.

In embodiments, the bioreactor assembly 100 is fluidically coupled to the perfusion system 800 via the one or more inlet ports 118 and the one or more outlet ports 119. Any suitable mechanism for fluidically coupling the components of the perfusion system 800 and allowing perfusion of the perfusate is contemplated and possible, including but not limited to, tubing (including rigid, semi-rigid, and flexible tubing) of various materials, such as polymers (i.e., TYGON®, polyurethane, fluorinated ethylene propylene), stainless steel, copper, steel, aluminum, plastics, silicone, or the like). In embodiments, the exterior facing inlet ports 118a and exterior facing outlet ports 119a are fluidically coupled to the interior chamber 104 via fluidic pathways in the lid assembly 200. In embodiments, the exterior facing inlet ports 118a and exterior facing outlet ports 119a are fluidically coupled to a fluid reservoir 802, such as via tubing, pumps, fluid movers, etc.

Referring now to FIG. 7A, a flow path of the perfusion system 800 is depicted. In embodiments, the perfusion system 800 may be operated at discrete times or may be operated continuously. The perfusion system 800 generally includes a fluid reservoir 802, a perfusion tubing system 804, and a pump 806. In embodiments, the fluid perfusion system 800 may include one or more sensors 814, a gas exchanger 810, a filtration system 805, and/or a sampling mechanism 812. The fluid reservoir 802 is generally configured to hold perfusate. The fluid reservoir 802 is fluidically coupled to the pump 806 and the bioreactor assembly 100 via the perfusion tubing system 804.

Generally, perfusate is placed in the fluid reservoir 802. In embodiments, the perfusion system 800 circulates perfusate through the system and its components. As used herein the term “perfusate” refers to any fluid capable of being perfused. In embodiments, perfusate includes, but is not limited to, decellularization fluid; cell culture media; recellularization fluid; solutions containing growth factors, glucose, nutritive agents, pharmaceutical compounds, immune modulating agents, coagulation factors, hormones, antimicrobial compounds, combinations thereof, and the like; buffers, such as phosphate buffered saline (PBS); cell differentiation media; blood; combinations thereof and the like. In embodiments, the perfusate may contain one or more cell types for recellularization.

The perfusate is then drawn into the perfusion tubing system 804 via the pump 806. Various parameters of the perfusion system 800, including fluid flow rate and pressure can be controlled by any suitable mechanism, including one or more pumps, valves, and the like. In embodiments, the mechanism is a pump 806. In embodiments, the pump 806 may be coupled to the perfusion tubing system 804 and used to control the flow of perfusate. Suitable pumps include peristaltic pumps, vacuum pumps, positive displacement pumps, diaphragm pumps, centrifugal pumps, piston pumps, axial pumps and the like.

Referring now to FIGS. 7A-7B, the perfusate flows through the perfusion tubing system 804 into the one or more inlet ports 118. In embodiments, the perfusate flows into a fluidic pathway via the exterior facing inlet port 118a, and exits the fluidic pathway into the interior chamber 104 of the bioreactor assembly 100 through an interior chamber facing outlet port 119a, depicted in FIGS. 1A-1B and 2B. In embodiments, the perfusion tubing system 804 extends through the lid assembly 200 and couples the one or more cannulations 700 to the perfusion system 800. The perfusate can then flow through the organ or organ scaffold 108 and exit into the interior chamber 104 of the bioreactor assembly 100. In embodiments, the perfusion system 800 is fluidically coupled to the organ or organ scaffold 108, such that perfusate flows into the organ or organ scaffold 108. As shown in FIG. 7B, in embodiments, the organ or organ scaffold 108 is cannulated such that the cannulations 700 fluidically couple the organ or organ scaffold 108 to the perfusion system 800. As used herein, “cannulation” refers to the insertion of a hollow tube (i.e. a cannula) into the organ. In embodiments, the organ or organ scaffold 108 is cannulated prior to being positioned within the bioreactor.

Referring jointly to FIGS. 1A, 5, and 7B, in embodiments, the organ or organ scaffold 108 is placed in a cradle 302 of the bioreactor assembly 100. In embodiments, the organ or organ scaffold 108 is cannulated prior to being placed with the bioreactor assembly 100. In other embodiments, the organ or organ scaffold 108 is cannulated within the bioreactor assembly 100. It is contemplated that one or more cannulations 700 can be placed using the robotic arm 502 and the ultrasound imaging unit 106 to automate placement of the cannulations 700. In embodiments, the one or more cannulations 700 are disposed within a naturally-occurring cavity or duct of the organ or organ scaffold 108. In embodiments, an organ may have a plurality of cannulations 700. The particular cannulation arrangement can vary according to the specific organ structure. For example, the organ may have 1, 2, 3, 4, 5, 6, 7, 8, 9, or 10 cannulations.

Referring back to FIG. 7A, perfusate exiting the bioreactor assembly 100 is drawn into the perfusion tubing system 804. In embodiments, perfusate flows out of the bioreactor assembly 100 via the one or more outlets 119. For example, perfusate can enter an interior chamber facing outlet port 119b, depicted in FIG. 2B, into a fluidic pathway, and flow into the perfusion tubing system 804 via an exterior facing outlet port 119a. In embodiments, the perfusate is then transported through the perfusion tubing system 804 into the fluid reservoir 802. In other embodiments, the perfusate is purged from the perfusion system 800 instead of being recycled into the reservoir. The perfusate may be purged using a valve, drain, or any other suitable mechanism. The determination of whether to recycle or purge the perfusate may be made by any number of criteria, including but not limited to, duration the perfusate has been in the perfusion system 800, data from the sampling mechanism 812 and/or sensors 814, discussed in further detail below, and/or transition between bioengineering phases.

In embodiments, the fluid reservoir 802 is capable of being refilled and/or drained, based upon the requirements of the stage of bioengineering or the requirements of the specific organ or organ scaffold 108. For example, during recellularization, removal of metabolic waste and/or addition of supplemental nutrients is necessary. During transition between bioengineering stages (e.g., decellularization to recellularization, recellularization to maturation, etc.) the perfusion system 800 may require a complete change of perfusate. In embodiments, the reservoir is fluidically coupled to a dispensing system (not shown), which may include one or more containers configured to hold one more components that can be used to adjust one or more cell culture parameters, including pH, glucose, salinity, mineral concentrations and the like. In embodiments, the one or more containers may be coupled to one or more pumps or may be gravity fed. The one or more pumps may be communicatively coupled to the one or more processors 904, discussed in further detail below, to regulate the amount of the one or more components that are dispensed in to the perfusate.

The perfusion system 800 may also include a filtration system 805. In embodiments, if the perfusate is recycled, the filtration system may be positioned such that the perfusate flows through the filter before being reintroduced into the fluid reservoir 802. The filtration system can be used to remove damaged cells and/or metabolic waste from the perfusate. Illustrative filtration systems include, but are not limited to, sequential graded filters, in-line filters, microfilters, membrane filters, exhaust filtration, and the like.

In embodiments, the perfusion system 800 is operated with additional equipment to optimize the perfusate, bioreactor assembly 100, and/or cell culture conditions. In embodiments, the fluid reservoir 802 is capable of maintaining the temperature of the perfusate at a specific temperature, or within a range of temperatures. In embodiments, the perfusate is kept at about 37° C. to mimic body temperature. Any suitable mechanism for maintaining the temperature is contemplated and possible. Illustrative, non-limiting examples include one or more thermal water jackets, heat pumps, Peltier devices, refrigeration cooling devices, resistive heating devices, and other temperature control devices within the reservoir. In some embodiments, the bioreactor assembly 100 may include the mechanism for maintaining the temperature of the perfusate. In embodiments, the bioreactor assembly 100 may include one or more additional mechanisms for maintaining the temperature of the perfusate. In embodiments, the bioreactor assembly 100 includes one or more resistive heating elements. In embodiments, the perfusion system 800 includes one or more resistive heating elements positioned near the bioreactor assembly 100 and/or one or more resistive heating elements at the fluid reservoir 802.

During different stages of bioengineering an organ or organ scaffold 108, oxygen and/or carbon dioxide may be key substrates for various biochemical processes. If dissolved oxygen levels are too low, growing cells may not accomplish necessary physiologic processes required to grow, proliferate, and/or differentiate. Conversely, oxygen concentrations within the human body are lower than atmospheric oxygen concentrations and some cell types have increased survival, proliferation, growth, and/or differentiation in environments with lower than atmospheric concentrations of oxygen. Additionally, excess oxygen may lead to the generation of reactive oxygen species, which can result in cellular dysfunction. Therefore, to maintain cell growth rate and nutrient uptake, dissolved oxygen levels may be kept within prescribed values, which may vary based on the organ or organ scaffold 108 being used, the cell type(s) used for recellularization, and/or the bioengineering stage.

Accordingly, the perfusion bioreactor system 900 may be configured to provide sufficient oxygen and/or gas exchange to the bioreactor assembly 100. In embodiments, such as shown in FIG. 7A, the perfusion system 800 can include a dissolved gas sensor 808 and a gas exchanger 810, communicatively coupled to the dissolved oxygen sensor and configured to replenish oxygen content in the perfusate. Any gas exchanger 810 is contemplated and possible. In embodiments, the gas exchanger 810 may also be configured to maintain a pressure, oxygen, carbon dioxide, or nitrogen concentration within the bioreactor assembly 100. Illustrative, non-limiting example of suitable gas exchangers include sparging systems, gas exchange systems, membrane oxygenators, photocatalytic oxygenators, thin-film oxygenators, airlift reactors, stirred tank reactors, and the like. While the gas exchanger 810 may be disposed within the interior chamber 104 of the bioreactor assembly 100, in such embodiments, particularly during recellularization, the perfusate must be calibrated to the appropriate oxygen levels prior to entering the bioreactor assembly 100 to avoid stressing the cells and/or organ or organ scaffold 108. In embodiments, the perfusion bioreactor system 900 may further include other gas dispensing systems, such as carbon dioxide and/or nitrogen dispensers to maintain the appropriate oxygen concentrations.

In embodiments, the perfusion system 800 may include a sampling mechanism 812. The sampling mechanism 812 includes an access site from which one or more perfusate samples can be collected without compromising the sterility of the system. These samples may be collected and analyzed to monitor nutrient consumption, to determine when perfusate exchange should occur, to determine the volume of perfusate to replace, to monitor levels of cell growth and/or proliferation, to monitor the health of the organ, and to assess biochemical profiling in functionality testing, discussed in greater detail below.

In addition to monitoring samples, for optimal bioengineering results the perfusion bioreactor system 900 may monitor a variety of parameters that can affect the bioengineering process. In embodiments, such as shown in FIGS. 7A and 7B, the perfusion bioreactor system 900 includes one or more sensors 814, configured to measure a plurality of parameters, including, but not limited to, pH, temperature (of the system and/or the organ or organ scaffold 108) humidity, pressure, capacitance, flow rate, glucose, salinity, metals, halogens, ammonia, oxygen, carbon dioxide, other dissolved gases, and the like. In embodiments, the sensors 814 may be disposed within the bioreactor assembly 100, within the perfusion system 800, and/or within the injection assembly 500. In embodiments, the sensors 814 are disposed within the perfusion tubing system 804. In embodiments, the sensors 814 are disposed within the fluid reservoir 802. In embodiments, the sensors 814 may be probes coupled to the lid assembly 200.

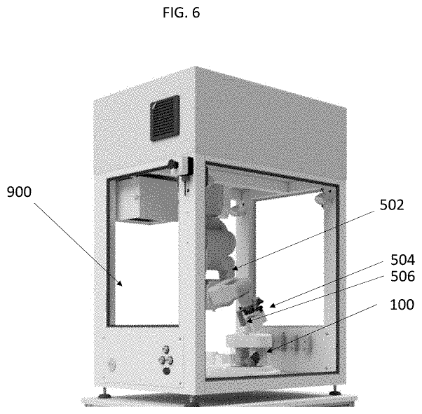

Referring again to FIGS. 5 and 6, the perfusion bioreactor system 900 generally includes an injection assembly 500. The injection assembly 500 generally includes a robotic arm 502, an injection tool 504, and a material dispensing mechanism 506. In embodiments, the injection assembly 500 is incorporated into a 3-D bioprinter as illustrated in FIG. 6, such as, for example a BioAssemblyBot®, as produced by Advanced Solutions Life Sciences, located in Louisville, Ky. However, it is noted that perfusion bioreactor system 900 may be used with any robotic assembly utilizing robotic injection tools, for example, robotic welding systems, robotic pick and place systems, robotic surgery systems, and the like.

Referring now to FIG. 6, the robotic arm 502 may be configured for various motions along a preprogrammed robot coordinate system. For example, the robotic arm 502 may be configured for 4-axis motion, 5-axis motion, 6-axis motion, 7-axis motion, or more. The robotic arm 502 may be configured to have an injection tool 504 attached thereto. For example, an injection tool 504 may be coupled at a distal end of the robotic arm 502.

Referring back now to FIG. 5, an exemplary injection tool 504 and dispensing mechanism 506 is depicted. In embodiments, the injection assembly 500 and dispensing mechanism 506 includes one or more of the BioAssemblyBot® Hands, as produced by Advanced Solutions Life Sciences, located in Louisville, Ky. However, it is noted that any injection tool 504 and dispensing mechanism 506 may be used with the robotic arm 502. In embodiments, the injection tool 504 includes a needle 508 and a syringe 510. In embodiments, the needle 508 is fluidically coupled to the syringe 510. In embodiments, the needle 508 is of a suitable length to deposit material to a desired location on the organ or organ scaffold 108 within a field of view of the ultrasound imaging unit 106. In embodiments, the syringe 510 holds or is configured to hold a material to be dispensed into the bioreactor assembly 100. In some embodiments, the robotic arm 502 may include additional tools other than the injection tool, including pick-and-place tools.

In embodiments, the dispensing mechanism 506 is configured to allow storage of the material to be dispensed. In embodiments, the storage is temperature controlled. In embodiments, the storage temperature ranges from 0° C. to 37° C. In embodiments, the dispensing mechanism 506 is responsible for mechanical actuation of the syringe to dispense the material. Automated use of the dispensing mechanism provides accurate control of injection rate and volume of the material dispensed.

In embodiments, the needle 508 and/or the injection tool 504 of the injection assembly 500 is placed within injection tool access port 214 defined by the lid assembly 200. In embodiments, the injection tool access port 214 may be in an open configuration to allow the needle 508 to access the interior chamber 104. In embodiments, the injection tool access port 214 is configured to automatically open to allow the injection tool 504 to be positioned to reach the organ or organ scaffold 108. In embodiments, while in the closed configuration, the injection tool access port 214 is covered by a lid seal to the lid assembly at the injection tool access port 214, which may be programmatically removed by the robotic arm 502 to place the injection tool access port 214 in the open configuration. In some embodiments, to place the injection tool access port 214 in the open configuration, the injection assembly 500 may pierce the membrane to access the interior chamber 104. In embodiments, the injection tool 504 is positioned to dispense the material onto and/or into the organ or organ scaffold 108, discussed in greater detail below.

In embodiments, the material to be dispensed includes one or more cell types for recellularizing the organ or organ scaffold 108 to generate a bioengineered organ. In embodiments, the one or more cell types are regenerative cells. Illustrative, non-limiting examples of regenerative cells include embryonic stem cells, inducible pluripotent stem cells, fetal stem cells, umbilical cord blood cells, tissue-derived stem cells, tissue-derived progenitor cells, bone marrow-derived stem cells, bone marrow-derived progenitor cells, blood-derived stem cells, blood-derived progenitor cells, mesenchymal stem cells (MSC), skeletal muscle-derived cells, adipose-derived stem cells, adipose-derived progenitor cells, multipotent adult progenitor cells (MAPC), multipotent adult stem cells, amniotic fluid-derived cells, urine-derived cells, cardiac stem cells (CSC), multipotent adult cardiac-derived stem cells, cardiac fibroblasts; cardiac microvasculature endothelial cells, aortic endothelial cells, bone marrow-derived stem cells, bone marrow mononuclear cells (BM-MNC), stromal cells, endothelial stem cells, vascular stem cells, vascular progenitor cells, endothelial progenitor cells (EPC), combinations thereof and the like. In embodiments, dispensing the cells includes dispensing through the injection assembly 500 and/or adding the one or more cell types to perfusate and perfusing the organ or organ scaffold 108.

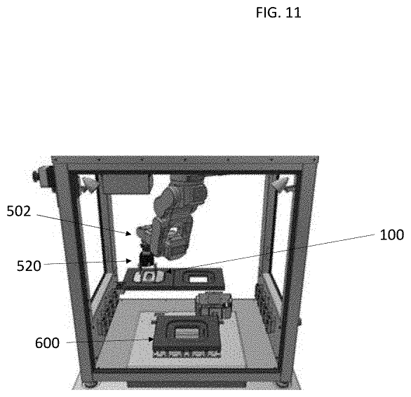

In embodiments, the base 600 may provide a nest into which the bioreactor assembly 100 may be place and/or recessed. In some embodiments, a bioengineering environment may include multiples bases 600 positioned throughout the environment to receive and hold the perfusion assembly. In some embodiments, a base 600 may be configured with sensors, ports or the like, to identify when a bioreactor assembly 100 is placed therein. In some embodiments, the base 600 may include RFID reader to read an RFID tag associated with the particular bioreactor assembly, which can be used to aid in transitioning between various stages of bioengineering. For example, a base 600 may output a signal that indicates to the perfusion bioreactor system 900 that the organ or organ scaffold 108 needs to undergo decellularization, recellularization or maturation processes. Upon completion of a process, the perfusion bioreactor system 900 may, using the robotic arm 502, pick up the bioreactor assembly 100 and move the bioreactor assembly to another location or base 600 for additional bioengineering processes, such as shown in FIG. 11.

In some embodiments, each base 600 may include associated non-transitory machine-readable instructions for a particular bioengineering process, such that positioning of a bioreactor assembly 100 within a particular base, may signal to the perfusion bioreactor system 900 to start a particular bio-engineering process. For example, in embodiments, a perfusion bioreactor system 900 may include an ingress base for transporting a bioreactor assembly 100 into a 3D bio-printing platform, a bio-printing platform base positioned within the 3D bio-printer, and an egress base for transporting the bioreactor assembly 100 out of the bio-printing platform. In embodiments, the ingress and/or egress nests may be mounted to moveable carts and the robotic arm 502, using a pick and place tool 520, may move the bioreactor assembly 100 from one base 600 to another depending on the stage of bioengineering.

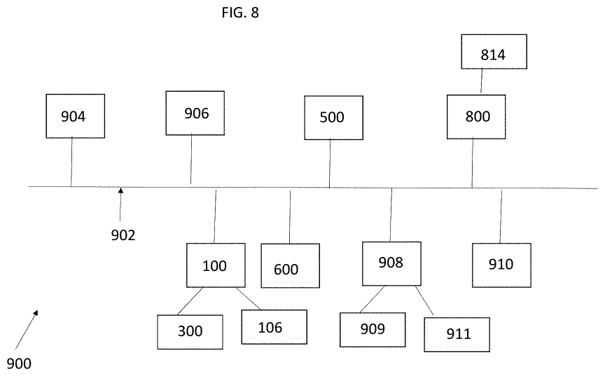

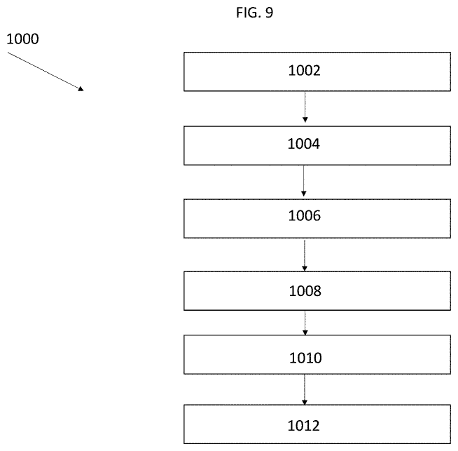

FIG. 8 schematically illustrates the perfusion bioreactor system 900 including one or more processors 904, the injection assembly 500, the perfusion system 800, the base 600, and/or the bioreactor assembly 100. It is noted that a greater or fewer number of modules may be included within the perfusion bioreactor system 900 without departing from the present disclosure. It is further noted that lines (e.g., communication path 902) within FIG. 9 are intended to show communication and not necessarily physical locations or proximities of modules relative to one or another. That is, modules of the present perfusion bioreactor system 900 may operate remotely from one another in a distributed computing environment.

In the depiction of FIG. 8 communication between the various components of the perfusion bioreactor system 900 may be provided over a communication path 902. As will be explained in greater detail herein, perfusion bioreactor system 900 may be configured to control operations of the robotic arm 502, the injection assembly 500, and the gimbal assembly 300 of the bioreactor assembly 100 to precisely position the injection tool 504 and/or the organ or organ scaffold 108 at specific locations for initiating a specific workflow process using one or more processors 904 to execute logic stored on one or more memory modules 906.

The one or more processors 904 may be communicatively coupled to the other components of the perfusion bioreactor system 900 over the communication path 902 that provides signal interconnectivity between the various components of the perfusion bioreactor system 900. As used herein, the term “communicatively coupled” means that coupled components are capable of exchanging data signals with one another such as, for example, electrical signals via conductive medium, electromagnetic signals via air, optical signals via optical waveguides, and the like.

Accordingly, the communication path 902 may be formed from any medium that is capable of transmitting a signal such as, for example, conductive wires, conductive traces, optical waveguides, or the like. In some embodiments, the communication path 902 may facilitate the transmission of wireless signals, such as WiFi, Bluetooth, and the like. Moreover, the communication path 902 may be formed from a combination of mediums capable of transmitting signals. In one embodiment, the communication path 902 comprises a combination of conductive traces, conductive wires, connectors, and buses that cooperate to permit the transmission of electrical data signals to components such as processors 904, memory modules 906, sensors 814, input devices, output devices, and communication devices. Accordingly, the communication path 902 may comprise a vehicle bus, such as for example a bus or the like. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium.

The one or more processors 904 may include any device capable of executing machine-readable instructions stored on a non-transitory computer readable medium. Accordingly, the processor may include a microcontroller, an integrated circuit, a microchip, a computer, and/or any other computing device.

The one or more memory modules 906 are communicatively coupled to the one or more processors 904 over the communication path 902. The one or more memory modules 906 may be configured as volatile and/or nonvolatile memory and, as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the perfusion bioreactor system 900 and/or external to the perfusion bioreactor system 900. The memory module 906 may be configured to store one or more pieces of logic to control the various components of the perfusion bioreactor system 900.

Embodiments of the present disclosure include logic stored on the one or more memory modules 906 as machine-readable instructions to perform an algorithm written in any programming language of any generation (e.g., 1GL, 2GL, 3GL, 4GL, and/or 5GL) such as in machine language that may be directly executed by the one or more processors 904, assembly language, obstacle-oriented programming (OOP), scripting languages, microcode, etc., that may be compiled or assembled into machine readable instructions and stored on a machine readable medium. Similarly, the logic may be written in a hardware description language (HDL), such as logic implemented via either a field-programmable gate array (FPGA) configuration or an application-specific integrated circuit (ASIC), and their equivalents. Accordingly, the logic may be implemented in any conventional computer programming language, as pre-programmed hardware elements, and/or as a combination of hardware and software components. As will be described in greater detail herein, machine-readable instructions stored on the one or more memory modules 906 allows the one or more processors 904 to, for example, process ultrasound imaging data to identify characteristics of the organ or organ scaffold 108 (such as via object recognition or other characteristics measurable or identifiable via ultrasound). The one or more processors 904 may further execute the machine-readable instructions to, based on the identified characteristics, begin an injection or recellularization protocol, alert a user to select a starting position, and/or adjust operating parameters of the robotic arm 502, injection tool 504, and/or gimbal assembly 300. As noted above, the embodiments described herein may utilize a distributed computing arrangement to perform any portion of the logic described herein.

In some embodiments, the perfusion bioreactor system 900 further includes an ultrasound analytics module 908 and a machine-learning module 910 for intelligently identifying characteristics on an organ scaffold 108 sufficient for initiating a recellularization process. As noted above, the ultrasound imaging unit 106 includes any unit configured to collect and transmit ultrasound imaging data and/or volumetric or spatial data collected by the ultrasound imaging unit 106. The ultrasound imaging unit 106 may be communicatively coupled to the one or more processors 904 via the communication path 902 to allow the one or more processors 904 to control the injection module 500 and/or the robotic arm 502 coupled thereto to selectively inject material into or onto the organ or organ scaffold 108. In embodiments, the ultrasound analytics module 908 has a tissue vision module 909 that determines from the ultrasound image data three dimensional volumetric and/or spatial image data of the organ or organ scaffold 108. The tissue vision module 909 provides an imaging solution for visualizing boundaries and internal structures of the organ or organ scaffold 108.

In embodiments, the ultrasound analytics module 908 includes a guided injection module 911. The guided injection module 911 may operate in conjunction with the tissue vision module 909 or independently. In embodiments, the two modules use real-time volumetric and spatial verification to provide robotic trajectory guidance to an injection assembly 500 during tissue/cell injection protocols, discussed in further detail below. In embodiments, the ultrasound imaging unit 106 is communicatively coupled to a display (e.g., a touch screen display), discussed in further detail below. In embodiments, a user can manually select an injection site from the display.

The ultrasound analytics module 908 is configured to at least apply data analytics and artificial intelligence algorithms and models to the images, including the volumetric and/or spatial image data, received from the ultrasound imaging unit 106. The machine-learning module 910 is configured for operating with such artificial intelligence algorithms and models, to continue to improve accuracy of said algorithms and models through application of machine learning. By way of example, and not as a limitation, the machine-learning module may include an artificial intelligence component to train and provide machine-learning capabilities to a neural network as described herein. In an embodiment, a convolutional neural network (CNN) may be utilized. The ultrasound analytics module and the machine-learning module may be communicatively coupled to the communication path 902 and the one or more processors 904. As will be described in further detail below, the one or more processors 904 may, using at least the ultrasound analytics module and/or the machine-learning module 910, process the input signals received from the perfusion bioreactor system 900 to adjust one or more conditions of the perfusate and/or bioreactor assembly 100 and/or extract information (e.g., ultrasound imaging data) from such signals.

For example, data stored within the one or more memory modules 906 and manipulated in the perfusion bioreactor system 900 as described herein may be utilized by the machine-learning module. In embodiments, the machine learning module 910 may leverage sensor information from the one or more sensors 814 to determine growth patterns, process steps, or the like. The machine-learning module may be able to leverage a cloud computing-based network configuration such as the cloud to apply Machine Learning and Artificial Intelligence as terms of art readily understood by one of ordinary skill in the art. This machine-learning module may be applied to and improve models that can be applied by the perfusion bioreactor system 900, to make it more efficient and intelligent in execution. As an example and not a limitation, the machine-learning module may include artificial intelligence components selected from the group consisting of an artificial intelligence engine, Bayesian inference engine, and a decision-making engine, and may have an adaptive learning engine further comprising a deep neural network-learning engine. It is contemplated and within the scope of this disclosure that the term “deep” with respect to the deep neural network learning engine is a term of art readily understood by one of ordinary skill in the art. In embodiments, to apply and improve upon an ultrasound guided model via machine-learning, volumetric and/or spatial image data from numerous organs and/or organ scaffolds may be recorded using the ultrasound imaging unit 106 during various stages of the bioengineering process and used by the machine-learning module to identify injection sites, improve positional accuracy of, improve injection accuracy and precision, and update the ultrasound guided model as the organ or organ scaffold 108 changes position and/or characteristics during different stages of bioengineering. In some embodiments, some organs or organ scaffolds may include purposely-created sites that are particularly suitable for initiating an injection procedure. The raw ultrasound images may then be split into individual images, which may then be annotated, e.g., by a user to indicate these sites, and used to train the model of the perfusion bioreactor system 900 to select appropriate sites to initiate workflow processes corresponding to recellularization. Using this technique, results can be incrementally improved over time by incorporating new data into the training process for the model.

In some embodiments, the machine-readable instructions, ultrasound analytics modules and/or the machine learning module are implemented via Tissue Structure and Information Modeling (TSIM®) 3D Design and Control Software, as produced by Advanced Solutions Life Sciences, located in Louisville, Ky.

Still referring to FIG. 8, the injection assembly 500 is communicatively coupled to the one or more processors 904 over the communication path 902. As will be described in greater detail herein, the one or more processors 904 may execute machine-readable instructions to control operation of the injection assembly 500. For example, the one or more processors 904 may execute machine-readable instructions such that the perfusion bioreactor system 900 can adjust operating parameters of the injection assembly 500 (e.g., speed, pressure, depth, angle, etc.), in response to data collected by the ultrasound imaging unit 106, the one or more sensors 814, and/or positioning of the organ or organ scaffold 108 with the gimbal assembly 300. The ultrasound imaging unit 106 may mounted such that the data provided by the ultrasound imaging unit 106 corresponds to the preprogrammed robot coordinate system of the injection assembly 500 to position the organ or organ scaffold using the gimbal assembly 300.

In embodiments, the gimbal assembly 300 is communicatively coupled to the one or more processors 904 via the communication path 902. As will be described in greater detail herein, the one or more processors 904 may execute machine-readable instructions to control operation of the gimbal assembly 300. In embodiments, the gimbal assembly 300 can position the cradle 302 (and the organ or organ scaffold 108 disposed thereon) for various stages of the bioengineering process, as may be determined based on ultrasound analytics module 908 and/or the machine-learning module 910, for example.

To effectively position the organ or organ scaffold 108 using the gimbal assembly 300, the one or more drive systems 400 are communicatively coupled to the one or more processors 904 over the communication path 902, such that the one or more processors 904 can execute logic to operate the one or more motors 404 to provide rotation about a first rotational axis and/or a second rotational axis to position the organ or organ scaffold for the bioengineering process.

In embodiments, the one or more sensors 814 used in evaluating the perfusate and/or the conditions of the bioreactor can send data to the one or more processors 904, which can execute logic relative to monitoring and/or adjusting the conditions of the perfusate or bioreactor assembly 100 using, for example, the additional equipment or the dispensing system. In embodiments, the perfusion bioreactor system monitors perfusate conditions to adapt to the needs of the organ or organ scaffold 108 or the stage of bioengineering. For example, as maturation proceeds, an organ or organ scaffold 108 may need increased levels of oxygen and/or nutrients. In such situations, the perfusion bioreactor system 900 can adjust the perfusion system 800 to better meet the needs of the organ or organ scaffold 108. For example, the perfusion bioreactor system 900 may monitor the pH of the perfusate. If the pH of the perfusate is outside a desired value (e.g., 7.4), the perfusion bioreactor system 900 can adjust the CO₂ input using the gas exchanger 810 and/or replace the perfusate. In embodiments, the perfusion bioreactor system 900 may monitor the turbidity of the perfusate. If the turbidity is outside of a desired range, the perfusion bioreactor system 900 can replace the perfusate and/or increase filtration.

FIG. 9 illustrates a flowchart depicting a method 1000 for automating a process of bioengineering an organ using one or more of the ultrasound imaging unit 106, the injection assembly 500 and/or the perfusion system 800. As described herein, the process of bioengineering an organ may include one or more stages including decellularization, cell culture, recellularization, maturation, and functionality testing, among others, though any number of stages may be included. Though steps are shown in a particular order or with a particular number of steps, a greater or fewer number of steps in varying orders are contemplated and possible without departing from the scope of the present disclosure.

The method 1000 at step 1002 includes placing an organ or organ scaffold 108 within the cradle 302 of the gimbal assembly 300. Step 1004 includes capturing volumetric and/or spatial image data of the organ or organ scaffold 108 using the ultrasound imaging unit 106 (e.g., in real time). In some embodiments, the perfusion bioreactor system 900 may be automatically initiated to begin capturing and processing image data of the organ or organ scaffold 108 once the bioreactor assembly 100 is powered on. At step 1006, the ultrasound image data may be analyzed, by the one or more processors 904 to detect one or more locations on the organ or organ scaffold 108 for a bioengineering process (e.g., recellularization) to be initiated. For example, the perfusion bioreactor system 900 may execute logic, such as object recognition logic (e.g., such as via a trained object recognition model) to identify anatomical regions (e.g., chambers, walls, valves, septums, ducts, sinuses, lobes, etc.) of the organ or organ scaffold 108 from the ultrasound imaging unit 106 to map the organ or organ scaffold 108. As noted above, the image data may be captured using the ultrasound imaging unit 106, and analyzed by the one or more processors 904, in real time to provide feedback to user or to the perfusion bioreactor system 900 of the detection of one or more initiation locations. In some embodiments, a user may be prompted by the perfusion bioreactor system 900 to select an initiation location.

Still referring to FIG. 9, once an initiation location has been identified, the perfusion bioreactor system 900 may operate with the one or more processors 904 to position the injection assembly 500, at step 1008. The injection tool 504 of the injection assembly 500 is placed within the injection tool access port 214 defined by the lid assembly 200, as shown in FIG. 5. To position the injection tool 504, the one or more processors 904 execute logic stored in the one or more memory modules 906 to move the robotic arm 502 of the injection assembly 500 such that the needle 508 of the injection tool 504 is in an identified injection position. In some embodiments, depending on the specific characteristics of the organ or organ scaffold 108 and/or the identified location (e.g., type of organ, depth of injection location, etc.) and/or the type of material being dispensed (e.g., cell type), the perfusion bioreactor system 900 may, with the one or more processors 904, automatically adjust operating parameters (e.g., pressure settings, speed settings, depth setting, angle settings, or the like) of the perfusion bioreactor system 900 to effectively execute the bioengineering process.

At step 1010, the perfusion bioreactor system 900 may, with the one or more processors 904, position the organ or organ scaffold 108 using the gimbal assembly 300. This may occur before, after, or simultaneous with positioning the injection assembly 500. To position the organ or organ scaffold 108, the one or more processors 904 execute logic stored in the one or more memory modules 906 to move the arm assembly 310 such that the initiation location of the organ or organ scaffold 108 is aligned with the needle 508 of the injection tool 504. In embodiments, the organ or organ scaffold 108 is maintained within the field of view of the ultrasound imaging unit 106 as it is moved by the gimbal assembly.

Once the initiation location and the injection position are aligned, the perfusion bioreactor system 900 initiates a bioengineering workflow at step 1012, and described in greater detail herein. For example, in a recellularization workflow, the one or more processors 904 execute logic stored in the one or more memory modules 906 to move the robotic arm 502 such that the needle 508 of the injection tool 504 dispenses material to the organ or organ scaffold 108. The one or more processors 904 execute logic stored in the one or more memory modules 906 to actuate the dispensing mechanism 506 to deposit the material stored in the syringe 510 onto or into the organ or organ scaffold 108. This process may then be repeated several times (e.g., 1 or more iterations, 2 or more iterations, 4 or more iterations, 10 or more iterations, 20 or more iterations, 30 or more iterations, etc.) with the one or more processors 904 making continuous adjustments of the gimbal assembly 300 and/or the injection assembly 500 based on the ultrasound analytics module or the machine-learning module. In embodiments, the gimbal assembly 300 repositions the organ or organ scaffold 108 and the injection tool assembly 500 adjusts positions to dispense material at each initiation location using ultrasound guidance.

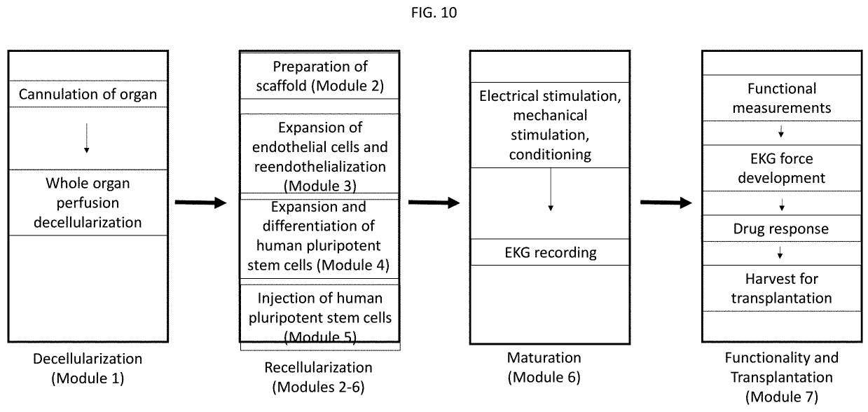

Referring now to FIG. 10 in conjunction with the previous figures, a flowchart summarizing the stages of generating a bioengineered organ for transplantation into a subject is depicted. In embodiments, the stages of the flowchart can be divided into seven primary modules, which can be automated by the perfusion bioreactor system 900.

The first module (Module 1) depicted in FIG. 10 is decellularization. In embodiments, the organ or organ scaffold 108 is cannulated prior to decellularization. As noted above, cannulations may be done manually or in some embodiments it is contemplated that cannulation or sub steps of cannulation may be performed via robotic control. For example, referring back to FIG. 7B, it is contemplated that the one or more processors 904 may execute the machine readable instructions from the software to move the gimbal assembly 300 to allow placement of the cannula 700 (depicted in FIG. 7B) such as with an end effector tool under ultrasound guidance using the ultrasound imaging unit 106. In embodiments, the cannulations 700 may be fluidically coupled to a fluid perfusion system 800, discussed in greater detail herein.

Still referring to FIG. 10, after the organ or organ scaffold 108 is cannulated, the organ is perfused with a decellularization fluid. The one or more sensors 814 used in evaluating the perfusate can send data to the one or more processors 904, which can execute logic relative to monitoring and/or adjusting the conditions of the decellularization fluid. In embodiments, the decellularization fluid includes one or more detergents, surfactants, chaotropic agents, osmotic agents, enzymes, enzyme inhibitors, vaso-active chemical solutions, and combinations thereof.

Illustrative, but non-limiting, examples of suitable detergents include ionic detergents, such as sodium dodecyl sulfate (SDS), deoxycholate, cholate, sarkosyl and the like; non-ionic detergents, such as polyethylene glycols (PEG), Triton X-100, n-Dodecyl-β-D-maltoside (DDM), digitonin, octyl-ß-D-glucoside, polysorbate 20, polysorbate 40, polysorbate 60, polysorbate 80 and the like; zwitterionic detergents, such as 3-((3-cholamidopropyl) dimethylammonio)-1-propanesulfonate (CHAPS), N-Dodecyl-N,N-(dimethylammonio)butyrate (DDMAB), miltefosine and the like; and combinations thereof.

Illustrative, non-limiting examples of enzymes include collagenases, matrix metalloproteinases, dispases, DNAses, proteases, and the like. Illustrative, non-limiting examples of enzymeinhibitors that can be used include, protease inhibitors, nuclease inhibitors, collagenase inhibitors, matrix metalloproteinase inhibitors, and the like. In embodiments, the decellularization fluid may include water that is osmotically incompatible with the cells such as RO water. In embodiments, the decellularization fluid can include other osmotic and/or chemical agents.

In embodiments, the cannulations are fluidically coupled to the perfusion system 800 in an arrangement configured to provide optimal decellularization conditions. For example, to decellularize a heart, a Langendorff perfusion system can be used with fluid entering into the aorta and exiting through the superior vena cava and/or other exit conduits. The cannulations can remain in place for the recellularization stage for the primary fluid or maintenance solution fluid circuit. To decellularize a liver, ingress can occur through the portal vein.

Still referring to FIG. 10, modules 2-5 all represent steps of the recellularization process. The second module (Module 2) is preparation of the scaffold, which may include, for example, placement of the organ scaffold 108 within the bioreactor assembly 100 and/or cannulation of the organ scaffold, which may be done manually or via automated control of the robotic arms having any suitable end effector coupled thereto. In embodiments, the organ scaffold 108 can be treated with growth enhancing medium to facilitate attachment of the cells to the ECM during recellularization, or the growth of cells during maturation.

In embodiments, recellularization comprises culturing cells to expand them prior to dispensing them onto an organ scaffold 108. In some embodiments, cells may not be cultured prior to introduction of an organ scaffold 108. Still referring to FIG. 10, the third module (Module 3) may include the expansion and/or introduction of endothelial cells. In embodiments, perfusate containing one or more types of endothelial cells is perfused through the bioreactor assembly 100 and perfusion system 800, thereby enabling functional reendothelialization of a vascular network in the organ or organ scaffold 108. The organ or organ scaffold 108 may be subjected to various types of fluid shear stress to aid in reendothelialization. Fluid shear stress may be adjusted for example, by adjusting the flow rate of perfusate through the perfusion system 800 and/or the pressure within the bioreactor assembly 100.

Reendothelialization generally includes the growth of endothelial tissue in an organ or organ scaffold 108. In some embodiments, additional components, such as growth factors may be introduced to the perfusion system 800 to assist in reendothelialization.

Still referring to FIG. 10, the fourth module (Module 4) includes the expansion and differentiation of human pluripotent stem cells. In some embodiments, the cells may be partially differentiated in culture or fully differentiated in culture prior to introduction to the organ or organ scaffold 108. Any suitable method for culturing and differentiating the one or more cell types is contemplated and possible.

The fifth module (Module 5) may include the injection of human pluripotent stem cells in to a decellularized organ scaffold 108. The perfusion bioreactor system 900 may automate the injection of stem cells as described above. For example, one or more cell types may be injected into the organ or organ scaffold 108 at a plurality of identified locations. In embodiments, different cell types can be injected into different locations of an organ scaffold 108. Alternatively, or in addition to injection, the one or more cell types may be perfused through the perfusion system 800.

The sixth module (Module 6) depicted in FIG. 10 includes maturation of the bioengineered organ. Once the organ scaffold 108 has been injected, the cells may begin to grow and expand. Upon completion of the injection workflow, the perfusion bioreactor system 900 may evaluate the data from the one or more sensors 814, the ultrasound imaging unit 106, control the sampling mechanism 812 and/or other variables (e.g., time) to determine the perfusate to be used. For example, expansion and/or differentiation media may be used after the injections are completed to induce growth and/or differentiation of the cells. If progenitor cells are used rather than mature cells, and then allowed to grow, the need for perfect seeding and engraftment is unnecessary. Further, as the organ matures, the perfusate may be switched from growth media to blood to prepare the bioengineered organ for transplantation. In embodiments, the blood is from the intended recipient.

In embodiments, the perfusion bioreactor system 900 can provide physical stimulation to the organ as it is maturing. For example, electronic stimulation can be provided to a recellularized heart, using for example, the electrodes 703, depicted in FIG. 7B. In embodiments, maturation of the recellularized organ 108 includes mechanical conditioning, such as by adjusting pressure and flow through or around the organ 108, which can be adjusted by the perfusion bioreactor system 900.

Still referring to FIG. 10, the seventh module (Module 7) may include bioanalytics and function testing to ensure that the organ is suitable for transplantation, at which point the organ is harvested and transplanted into a subject. Functional testing may include, for example, clearance kinetics, glucose metabolism, metabolite monitoring, and the like. In embodiments, functionality testing may occur utilizing the sampling mechanism 812 previously described.

In embodiments, the systems and bioreactors described herein automate at least one of Modules 1-7. In embodiments, the systems and bioreactors automate Module 1, Module 2, Module 3, Module 4, Module 5, Module 6, or Module 7. In embodiments, the systems and bioreactors automate a plurality of modules selected from Module 1, Module 2, Module 3, Module 4, Module 5, Module 6, and/or Module 7. In embodiments, the systems and bioreactors automate any combination of two, three, four, five, or six of the modules. In embodiments, the systems and bioreactors automate at least Module 5 and Module 6.

Embodiments may be further described with respect to the following aspects:

In a first aspect, alone or in combination with any other aspects herein, the present disclosure relates to a bioreactor assembly comprising: a housing defining an interior chamber; a lid assembly removably coupled to the housing and enclosing the interior chamber; a gimbal assembly disposable within the interior chamber, the gimbal assembly comprising: a cradle configured to hold an organ or organ scaffold; and an arm assembly configured to move the cradle between a plurality of positions; and an ultrasound imaging unit positioned to capture volumetric and/or spatial data of the organ or organ scaffold.

In a second aspect, alone or in combination with any other aspects herein, the present disclosure relates to a bioreactor assembly wherein the lid assembly defines an injection tool access port for receiving an injection tool therethrough.

In a third aspect, alone or in combination with any other aspects herein, the present disclosure relates to a bioreactor assembly, wherein the lid defines one or more fluidic pathways therethrough extending from an exterior facing inlet port to an interior chamber facing inlet port.

In a fourth aspect, alone or in combination with any other aspects herein, the present disclosure relates to a bioreactor assembly wherein the arm assembly comprises a first joint assembly configured to rotate the cradle about a first rotational axis and a second joint assembly configured to rotate the cradle about a second rotational axis different from the first rotational axis.

In a fifth aspect, alone or in combination with any other aspects herein, the present disclosure relates to a bioreactor assembly wherein the arm assembly comprises a first motor coupled to the first joint assembly and a second motor coupled to the second joint assembly, wherein each motor is independently operable from one another.

In a sixth aspect, alone or in combination with any other aspects herein, the present disclosure relates to a bioreactor assembly, wherein the arm assembly is coupled to the lid assembly.

In a seventh aspect, alone or in combination with any other aspects herein, the present disclosure relates to a bioreactor assembly, wherein the ultrasound imaging unit is coupled to the lid assembly.

In an eighth aspect, alone or in combination with any other aspects herein, the present disclosure relates to a bioreactor assembly, wherein the cradle comprises a connector end connecting the cradle to the arm assembly and a tissue support surface extending from the connector end and positioned to support the organ or organ scaffold thereon.

In a ninth aspect, alone or in combination with any other aspects herein, the present disclosure relates to a bioreactor assembly, wherein the arm assembly comprises a tissue mount configured to receive and support at least a portion of the organ or organ scaffold.

In a tenth aspect, alone or in combination with any other aspects herein, the present disclosure relates to a bioreactor assembly wherein the tissue mount defines a channel extending therethrough configured to receive tubing from the lid assembly to the tissue or organ scaffold.

In an eleventh aspect, alone or in combination with any other aspects herein, the present disclosure relates to a bioreactor assembly having one or more sensors positioned within at least one of the lid assembly or the housing, the one or more sensors comprising one or more of a pressure sensor, pH sensor, turbidity sensor, dissolved gas sensor, or a temperature sensor.

In a twelfth aspect, alone or in combination with any other aspects herein, the present disclosure relates to a perfusion bioreactor system comprising: a bioreactor comprising: a housing defining an interior chamber; a lid assembly removably coupled to the housing and enclosing the interior chamber and comprising one or more inlet ports and one or more outlet ports; a gimbal assembly disposable within the interior chamber, the gimbal assembly comprising: a cradle configured to hold an organ or organ scaffold; and an arm assembly configured to move the cradle between a plurality of positions; and an ultrasound imaging unit positioned to capture volumetric and/or spatial image data of the organ or organ scaffold; a fluid perfusion system, fluidically coupled to the bioreactor, comprising: a fluid reservoir configured to hold a perfusate; and a pump fluidically coupling the one or more inlet ports to the fluid reservoir; an injection assembly configured to dispense material to the organ or organ scaffold within the chamber; one or more processors communicatively coupled to the injection assembly, the ultrasound imaging unit, and the gimbal system, one or more memory modules communicatively coupled to the one or more processors, and machine-readable instructions stored on the one or more memory modules that, when executed by the one or more processors, cause the perfusion bioreactor system to: collect volumetric and/or spatial image data of the organ or organ scaffold using the ultrasound imaging unit; identify one or more initiation locations on the organ or organ scaffold within the volumetric and/or spatial image data; position the organ or organ scaffold using the gimbal assembly such that the one or more initiation locations are aligned with the injection assembly.

In a thirteenth aspect, alone or in combination with any other aspects herein, the present disclosure relates to a perfusion bioreactor system, wherein the machine-readable instructions, when executed by the one or more processors further cause the perfusion bioreactor system to initiate a bioengineering workflow, wherein said bioengineering workflow is a recellularization procedure comprising a plurality of injections material injections.

In a fourteenth aspect, alone or in combination with any other aspects herein, the present disclosure relates to a perfusion bioreactor system, wherein the machine-readable instructions, when executed by the one or more processors further cause the injection assembly to dispense material onto the organ or organ scaffold.

In a fifteenth aspect, alone or in combination with any other aspects herein, the present disclosure relates to a perfusion bioreactor system, wherein the material is cellular material.

In a sixteenth aspect, alone or in combination with any other aspects herein, the present disclosure relates to a method of recellularizing an organ scaffold, the method comprising placing the organ within a bioreactor assembly; collecting, with a processor, volumetric and/or spatial image data of the organ scaffold using an ultrasound imaging unit communicatively coupled to the processor; processing, with the processor, the volumetric and/or spatial image data to identify one or more initiation locations; controllably adjusting the bioreactor assembly communicatively coupled to the processor with the processor to position the organ scaffold within the to provide access to the one or more initiation locations; and dispensing or injecting a cellular material onto or into the organ scaffold.

In a seventeenth aspect, alone or in combination with any other aspects herein, the present disclosure relates to a method of recellularizing an organ scaffold wherein the cellular material is dispensed using an injection assembly communicatively coupled to the processor.

In an eighteenth aspect, alone or in combination with any other aspects herein, the present disclosure relates to a method of recellularizing an organ scaffold further comprising automatically adjusting operating parameters of the injection assembly with the processor in response to the volumetric and/or spatial image data.

In a nineteenth aspect, alone or in combination with any other aspects herein, the present disclosure relates to a method of recellularizing an organ scaffold, wherein the volumetric and/or spatial image data is collected in real-time.

In a twentieth aspect, alone or in combination with any other aspects herein, the present disclosure relates to a method of recellularizing an organ scaffold wherein: the bioreactor assembly comprises a bioreactor assembly of any of claims 1-11 and the step of dispensing the cellular material onto or into the organ scaffold comprises opening an injection tool access port formed within the lid.

Bioreactor assemblies and bioreactor perfusion systems of the present disclosure enable automated bioengineering process that result in higher levels of precision, speed, and better outcomes for organ harvesting. Using ultrasound guidance and precision placement of the organ scaffold during recellularization provides better selection of initiation sites and better adaptation of injections based on the specifics of the organ. By completing various stages of bioengineering within a single bioreactor assembly, contamination and stress on the organ or organ scaffold can be reduced.

Unless otherwise defined, all technical and scientific terms used herein have the same meaning as commonly understood by one of ordinary skill in the art to which the claimed subject matter belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that terms like “preferably,” “commonly,” and “typically” are not utilized herein to limit the scope of the appended claims or to imply that certain features are critical, essential, or even important to the structure or function of the claimed subject matter. Rather, these terms are merely intended to highlight alternative or additional features that may or may not be utilized in a particular embodiment.

What is claimed is:

1. A bioreactor assembly comprising:
a housing defining an interior chamber;
a lid assembly removably coupled to the housing and enclosing the interior chamber;
a gimbal assembly disposable within the interior chamber, the gimbal assembly comprising:
a cradle configured to hold an organ or organ scaffold; and
an arm assembly configured to move the cradle between a plurality of positions; and
an ultrasound imaging unit positioned to capture volumetric and/or spatial data of the organ or organ scaffold.

2. The bioreactor assembly of claim 1, wherein the lid assembly defines an injection tool access port for receiving an injection tool therethrough.

3. The bioreactor assembly of claim 1, wherein the lid defines one or more fluidic pathways therethrough extending from an exterior facing inlet port to an interior chamber facing inlet port.

4. The bioreactor assembly of claim 1, wherein the arm assembly comprises a first joint assembly configured to rotate the cradle about a first rotational axis and a second joint assembly configured to rotate the cradle about a second rotational axis different from the first rotational axis.

5. The bioreactor assembly of claim 4, wherein the arm assembly comprises a first motor coupled to the first joint assembly and a second motor coupled to the second joint assembly, wherein each motor is independently operable from one another.

6. The bioreactor assembly of claim 4, wherein the arm assembly is coupled to the lid assembly.

7. The bioreactor assembly of claim 1, wherein the ultrasound imaging unit is coupled to the lid assembly.

8. The bioreactor assembly of claim 1, wherein the cradle comprises a connector end connecting the cradle to the arm assembly and a tissue support surface extending from the connector end and positioned to support the organ or organ scaffold thereon.

9. The bioreactor assembly of claim 1, wherein the arm assembly comprises a tissue mount configured to receive and support at least a portion of the organ or organ scaffold.

10. The bioreactor assembly of claim 9, wherein the tissue mount defines a channel extending therethrough configured to receive tubing from the lid assembly to the tissue or organ scaffold.

11. The bioreactor assembly of claim 1, further comprising one or more sensors positioned within at least one of the lid assembly or the housing, the one or more sensors comprising one or more of a pressure sensor, pH sensor, turbidity sensor, dissolved gas sensor, or a temperature sensor.

12. A perfusion bioreactor system comprising:
a bioreactor comprising:
a housing defining an interior chamber;
a lid assembly removably coupled to the housing and enclosing the interior chamber and comprising one or more inlet ports and one or more outlet ports;
a gimbal assembly disposable within the interior chamber, the gimbal assembly comprising:
a cradle configured to hold an organ or organ scaffold; and
an arm assembly configured to move the cradle between a plurality of positions; and
an ultrasound imaging unit positioned to capture volumetric and/or spatial image data of the organ or organ scaffold;
a fluid perfusion system, fluidically coupled to the bioreactor, comprising:
a fluid reservoir configured to hold a perfusate; and
a pump fluidically coupling the one or more inlet ports to the fluid reservoir
an injection assembly configured to dispense material to the organ or organ scaffold within the chamber;
one or more processors communicatively coupled to the injection assembly, the ultrasound imaging unit, and the gimbal system,
one or more memory modules communicatively coupled to the one or more processors, and
machine-readable instructions stored on the one or more memory modules that, when executed by the one or more processors, cause the perfusion bioreactor system to:
collect volumetric and/or spatial image data of the organ or organ scaffold using the ultrasound imaging unit;
identify one or more initiation locations on the organ or organ scaffold within the volumetric and/or spatial image data;
position the organ or organ scaffold using the gimbal assembly such that the one or more initiation locations are aligned with the injection assembly.

13. The perfusion bioreactor system of claim 12, wherein the machine-readable instructions, when executed by the one or more processors further cause the perfusion bioreactor system to initiate a bioengineering workflow, wherein said bioengineering workflow is a recellularization procedure comprising a plurality of injections material injections.

14. The perfusion bioreactor system of claim 12, wherein the machine-readable instructions, when executed by the one or more processors further cause the injection assembly to dispense material onto the organ or organ scaffold.

15. The perfusion bioreactor system of claim 14, wherein the material is cellular material.

16. A method of recellularizing an organ scaffold, the method comprising
placing the organ within a bioreactor assembly;
collecting, with a processor, volumetric and/or spatial image data of the organ scaffold using an ultrasound imaging unit communicatively coupled to the processor;
processing, with the processor, the volumetric and/or spatial image data to identify one or more initiation locations;
controllably adjusting the bioreactor assembly communicatively coupled to the processor with the processor to position the organ scaffold within the to provide access to the one or more initiation locations; and
dispensing or injecting a cellular material onto or into the organ scaffold.

17. The method of claim 16, wherein the cellular material is dispensed using an injection assembly communicatively coupled to the processor.

18. The method of claim 17, further comprising automatically adjusting operating parameters of the injection assembly with the processor in response to the volumetric and/or spatial image data.

19. The method of claim 16, wherein the volumetric and/or spatial image data is collected in real-time.

20. The method of claim 16, wherein:
the bioreactor assembly comprises a bioreactor assembly of any of claims 1-11
and the step of dispensing the cellular material onto or into the organ scaffold comprises opening an injection tool access port formed within the lid.
